(19) United States
(12) Patent Application Publication
(10) Pub. No.: US 2024/0226576 A1
(43) Pub. Date: Jul. 11, 2024

(54) HEART SOUNDS TO OPTIMIZE CONDUCTION SYSTEM PACING

(71) Applicant: Cardiac Pacemakers, Inc., St. Paul, MN (US)

(72) Inventors: Pramodsingh Hirasingh Thakur, Woodbury, MN (US); Kevin Chu, Shoreview, MN (US); Viktoria A. Averina, Shoreview, MN (US)

(21) Appl. No.: 18/391,355

(22) Filed: Dec. 20, 2023

Related U.S. Application Data
(60) Provisional application No. 63/438,085, filed on Jan. 10, 2023.

(51) Int. Cl. A61N 1/368; A61N 1/365; A61N 1/372

(52) U.S. Cl. CPC A61N 1/368; A61N 1/36578; A61N 1/36585; A61N 1/37247; A61N 1/371

(57) ABSTRACT

Systems and methods for electrically stimulating a patient's cardiac conduction system at selected locations based on heart sounds are disclosed. A medical-device system includes a data receiver to receive heart sound information sensed from a patient, an electrostimulation circuit to deliver electrostimulation to the cardiac conduction system, and a controller circuit to generate, from the heart sound information sensed in response to the electrostimulation being delivered respectively to two or more candidate locations at or near the cardiac conduction system, heart sound metrics respectively for the two or more candidate locations. Based on the heart sound metrics, the controller circuit selects at least one of the candidate locations as a pacing location for subsequent electrostimulation. Electrostimulation can be delivered to the selected pacing location to restore cardiac synchrony.

### CLAIM OF PRIORITY

This application claims the benefit of U.S. Provisional Application No. 63/438,085, filed on Jan. 10, 2023, which is hereby incorporated by reference in its entirety.

### TECHNICAL FIELD

This document relates generally to medical systems, and more particularly, to systems, devices and methods for pacing a subject's cardiac conduction system at locations determined based on heart sounds.

### BACKGROUND

The heart is the center of a person's circulatory system. It includes an electro-mechanical system performing two major pumping functions. The left portions of the heart, including the left atrium (LA) and left ventricle (LV), draw oxygenated blood from the lungs and pump it to the organs of the body to provide the organs with their metabolic needs for oxygen. The right portions of the heart, including the right atrium (RA) and right ventricle (RV), draw deoxygenated blood from the body organs and pump it to the lungs where the blood gets oxygenated. These pumping functions result from contractions of the myocardium (cardiac muscles). In a normal heart, the sinoatrial (SA) node, the heart's natural pacemaker, generates electrical pulses, called action potentials, which propagate through natural electrical conduction pathways known as His-Purkinje system to various regions of the heart to excite the myocardial tissue of the heart. For example, the action potentials originated from the SA node propagate through the atrioventricular (AV) node, the His bundle (also known as Bundle of His), the bundle branches, and Purkinje fibers to reach the ventricular myocardium, resulting in coordinated contractions in both ventricles.

Coordinated delays in the propagation of the action potentials in a normal electrical conduction system cause the various portions of the heart to contract in synchrony to result in efficient pumping functions. A blocked or otherwise abnormal electrical conduction and/or deteriorated myocardium may cause dyssynchronous contraction of the heart, resulting in poor hemodynamic performance, including a diminished blood supply to the heart and the rest of the body. For example, an abnormal delay in the transmission of the action potentials in the His bundle may cause irregular or dyssynchronous contractions of the ventricles.

Artificial cardiac pacing system have been used to rectify cardiac dyssynchrony and to improve hemodynamic performance. The artificial cardiac pacing system may provide electrical stimulations to one or more portions of the heart such as to restore normal functioning of the heart to a certain extent. For example, right ventricular pacing via electrodes implanted in the apex of the RV have been used in both single ventricular and biventricular (BiV) pacing. RV apical pacing directly excites the ventricular myocardium in an unnatural way involving slow muscle-muscle contraction. Normally, ventricular excitation involves rapid coordinate activation via action potentials that are propagated through the natural conduction pathways. Studies have shown that, in some patients, the cardiac dyssynchrony induced by RV apical pacing, partially due to the interventricular delay in impulse propagation to the left ventricle, can result in adverse ventricular changes including enlarged ventricles and heart failure when delivered over the long term. Moreover, permanent changes in myocardial perfusion and structure may develop over time in these patients, which may further decrease cardiac output and deteriorate ventricular function. BiV pacing involves RV pacing via one lead, and LV pacing via another lead, and has been demonstrated to restore synchronized contraction of both ventricles. However, the potential adverse impact on ventricular function produced by the RV apical pacing may still exist in BiV pacing. Muscle-muscle activation via epicardial LV pacing is also known to result in a less vigorous contraction.

Heart sounds are generally associated with mechanical vibration of a heart and blood flow through the heart. Heart sounds recur with each cardiac cycle and are separated and classified according to the activity associated with the vibration. Historically, heart sounds were assessed by humans and thus only audible portion of the vibration was used. Devices can now assess full spectrum of cardiac vibrations which include both audible and subaudible components, thus the term “sound” in this document refers to the full spectrum of vibrations. Typically, heart sounds sensed from a subject may include several components within a cardiac cycle, including a first (S1), a second (S2), a third (S3), or a fourth (S4) heart sound. S1 is associated with the vibrations produced by the heart during closure of the atrioventricular (mitral and tricuspid) valves as the ventricular pressures exceed atrial pressures at the beginning of systole. S2 is produced by closure of the aortic and pulmonay valves, and marks the beginning of diastole. S3 is produced by early diastolic vibrations corresponding to passive ventricular filling during diastole, when the blood rushes into the ventricles. S4 is produced by late diastolic vibrations corresponding to active ventricular filling when the atria contract and push the blood into the ventricles. In a healthy subject, S3 is usually faint and S4 is rarely audible. However, a pathologic S3 or S4 may be higher pitched and louder.

Heart sounds have been used to assess cardiac systolic and diastolic functions. Systole is the contraction or a period of contraction of the heart that causes blood to be forced out of the heart such as the ventricles and into the aorta and pulmonary artery. Diastole is the relaxation or a period of relaxation of the heart during which the blood flows back into the heart such as the ventricles. Patients with cardiac diseases may have deteriorated systolic or diastolic functions. For example, congestive heart failure (CHF) occurs when the heart is unable to supply enough blood to maintain a healthy physiologic state.

### Overview

Hemodynamic response to cardiac pacing may depend on many factors, including pacing site selection and pacing configurations. Many patients receiving cardiac pacing therapy have an intact His bundle and intact cardiac conduction system (e.g., the His-Purkinje system) in the ventricles, and therefore having normal ventricular activation. Conventional cardiac pacing such as long-term RV apical pacing (i.e., pacing at RV apex) may cause a decrease in cardiac output and efficiency due to the uncoordinated contraction sequence. This dyssynchrony may eventually cause adverse long-term effects. Dyssynchronous contraction of the ventricles occurs during conventional RV pacing because the activation sequence may be much slower and propagate slowly from the right to the left ventricle across the interventricular septum, thereby causing ventricular dyssynchrony. This sequence of activation results in an uncoordinated contraction, which does not occur during biventricular activation through the natural conduction system of the heart. The cells of the natural conduction system may propagate an activation signal about four times faster than working myocardium. Pacing a portion of the natural cardiac conduction system (e.g., the His bundle, left or right bundle branches, fascicles, or Purkinje fibers, among other conductive cardiac tissue) is an alternative to conventional ventricular pacing. Conduction system pacing (CSP), such as His-bundle pacing (HBP), septal pacing such as left bundle branch pacing (LBBP) or right bundle branch pacing (REBP) at a septal region, or pacing at other parts the His-Purkinje conduction system, may activate the heart's natural conduction system, restore or improve cardiac synchrony, and reduce or even eliminate potential long-term harmful hemodynamic effects associated with conventional RV apical pacing.

However, when not being successful, the CSP may not adequately restore cardiac synchrony. For example, HBP may lose its ability to activate (or capture) the His bundle or neighboring conductive tissue, but only activates the para-Hisian myocardium. Stimulating muscles near the His bundle may cause dyssynchronous patterns similar to RV apical pacing. This undesirable outcome is referred to as para-Hisian capture. Simultaneous capture of the His bundle and para-Hisian muscle (also known as a non-selective His-bundle capture) may be as clinically effective as the His-bundle only capture (also known as a selective His-bundle capture, as the resultant ventricular activation and contraction is dominated by the more rapid conducting Purkinje system. Another undesirable outcome of HBP is known as a complete loss of capture (LOC), where the HBP pulses capture neither the para-Hisian myocardium nor the His bundle.

The ability of CSP to restore cardiac synchrony may also be dependent on the pacing site relative to the blockage site along the His-Purkinje system, such as at the His bundle or a bundle branch. Ventricular dyssynchrony in many CHF patients may be attributed to various degrees of left bundle branch block (LBBB), which causes delayed LV depolarization lagging behind RV depolarization. Effective propagation of the action potentials through the His-Purkinje system to restore cardiac synchrony may be achieved only if the HBP pulses are delivered distal to the blockage site. If the HBP pulses are delivered proximal to the blockage site, even if the proximal portion of the His bundle is activated, the action potential cannot bypass the blockage and propagate to the ventricles through the His-Purkinje system. Consequently, no cardiac synchrony may be restored.

Pacing site are conventionally determined by analyzing cardiac electrical signals such as surface electrocardiogram (ECG). ECG parameters such as QRS width has been used to quantify a degree of dyssynchrony and to identify an “optimal” or a desired pacing site at which the pacing would result in a shortening of QRS width. However, complex computation is generally required to have a robust and accurate QRS width measurement, which can be timing consuming. Stability of QRS width measurement may vary significantly in some post-implant patients. Additionally, while QRS width or other ECG parameters are indicative of or correlated to electrical dyssynchrony (or a restoration to synchrony), the QRS width and the ECG parameters may not adequately indicate or correlate to mechanical dyssynchrony between left and right ventricles or various portions of a ventricle. The present inventors have recognized a technical challenge in cardiac conduction system pacing, particularly more effective and efficient techniques to identify proper pacing site(s) at or near the patient's cardiac conduction system and a depth of insertion of a pacing electrode or pacing lead therein to reliably capture the conductive tissue and restore ventricle synchrony.

The present document describes an improved technique to optimize CSP pacing site and pacing parameters based on heart sound information sensed from a patient in response to a sequence of test CSP delivered receptively at a plurality of candidate locations at or near the patient's cardiac conduction system (e.g., the His-Purkinje system). An exemplary medical system includes a data receiver circuit to receive heart sound information from a patient, an electrostimulation circuit to deliver electrostimulation to a location at or near the patient's cardiac conduction system, and a controller circuit. The controller circuit can generate heart sound metrics, respectively for two or more candidate locations at or near the His-Purkinje system, using heart sound information sensed in response to the electrostimulation being delivered respectively to the two or more candidate locations, and select at least one of the candidate locations as a pacing location for subsequent electrostimulation based at least in part on the generated heart sound metrics. Electrostimulation may be delivered at the selected pacing location to restore cardiac synchrony or to improve cardiac performance.

Example 1 is a medical-device system, comprising: a data receiver circuit configured to receive heart sound information sensed from a patient; an electrostimulator configured to provide electrostimulation to a location at or near a cardiac conduction system of the patient; and a controller circuit configured to: generate, from the heart sound information sensed from the patient in response to the electrostimulation being delivered respectively to two or more candidate locations at or near the cardiac conduction system, heart sound metrics respectively for the two or more candidate locations; and select at least one of the candidate locations as a pacing location for subsequent electrostimulation based at least in part on the generated heart sound metrics.

In Example 2, the subject matter of Example 1 optionally includes the controller circuit that can be configured to select the pacing location for subsequent electrostimulation from the two or more candidate locations based on a comparison of the generated heart sound metrics for the two or more candidate locations.

In Example 3, the subject matter of any one or more of Examples 1-2 optionally includes the controller circuit that can be configured to: generate a reference heart sound metric using heart sound information sensed from the patient without the electrostimulation being delivered to any of the two or more candidate locations; and select the pacing location for subsequent electrostimulation based at least in part on a comparison of the generated heart sound metrics to the reference heart sound metric.

In Example 4, the subject matter of Example 3 optionally includes the controller circuit that can be configured to select the pacing location for subsequent electrostimulation that corresponds to a difference between the generated heart sound metric in response to the electrostimulation and the reference heart sound metric exceeding a threshold value.

In Example 5, the subject matter of any one or more of Examples 1-4 optionally includes the electrostimulator that can be electrically coupled to one or more electrodes positionable at two or more candidate locations in a His-bundle or septal region of the heart to deliver the electrostimulation therein, wherein the selected pacing location for subsequent electrostimulation includes a selected His-bundle or septal site. In some examples, the two or more candidate locations in the His-bundle or septal region include two or more candidate locations at or near one or more of a left bundle branch or a right bundle branch of the heart.

In Example 6, the subject matter of Example 5 optionally includes the one or more electrodes that can be associated with a lead having a proximal end coupled to the electrostimulator and a distal tip configured to be inserted into the His-bundle or septal region, wherein the selected pacing location for subsequent electrostimulation includes a selected depth of insertion of the distal tip of the lead.

In Example 7, the subject matter of any one or more of Examples 1-6 optionally includes the heart sound metric that can include an S1 heart sound intensity, wherein the controller circuit is configured to select the pacing location for subsequent electrostimulation with a corresponding S1 heart sound intensity exceeding a threshold.

In Example 8, the subject matter of any one or more of Examples 1-7 optionally includes the data receiver circuit that can be configured to receive cardiac electrical activity information sensed from the patient, wherein the controller circuit is configured to: generate, for each of the two or more candidate locations, the heart sound metric including a pre-ejection period further using the received cardiac electrical activity information; and select the pacing location for subsequent electrostimulation with a corresponding pre-ejection period falling below a threshold.

In Example 9, the subject matter of any one or more of Examples 1-8 optionally includes the heart sound metric that can include a split S1 pattern, wherein the controller circuit is configured to select the pacing location for subsequent electrostimulation that corresponds to a detection of the split S1 pattern or an occurrence rate of the split S1 pattern exceeding a threshold.

In Example 10, the subject matter of any one or more of Examples 1-9 optionally includes the data receiver circuit that can be configured to receive respiration information sensed from the patient, the respiration information including an inspiration phase and an expiration phase in a respiration cycle, wherein the controller circuit is configured to: generate, for each of the two or more candidate locations, the heart sound metric including a paradoxical split S2 pattern characterized by a presence of split S2 during the expiration phase and an absence of split S2 during the inspiration phase in the respiration cycle; and select the pacing location for subsequent electrostimulation that corresponds to a non-detection of the paradoxical split S2 pattern or an occurrence rate of the paradoxical split S2 pattern falling below a threshold.

In Example 11, the subject matter of any one or more of Examples 1-10 optionally includes the controller circuit that can be further configured to: generate heart sound metrics using the heart sound information sensed from the patient in response to the electrostimulation being delivered at the selected pacing location in accordance with varying pacing dosage or timing parameter values; and determine a pacing dosage or timing parameter value based at least in part on the generated heart sound metrics corresponding to the varying pacing dosage or timing parameter values.

In Example 12, the subject matter of any one or more of Examples 1-11 optionally includes the selected pacing location being a first selected pacing location, wherein the data receiver circuit is configured to receive cardiac electrical activity information, wherein the controller circuit is configured to select a second pacing location from the two or more candidate locations using the received cardiac electrical activity information, and to confirm the first selected pacing location using the second selected pacing location.

In Example 13, the subject matter of any one or more of Examples 1-12 optionally includes the controller circuit that can be configured to generate a control signal to a user interface to display in real time the generated heart sound metrics while the electrostimulation is being delivered respectively to the two or more candidate locations.

In Example 14, the subject matter of any one or more of Examples 1-13 optionally includes the controller circuit that can be configured to generate a control signal to a user interface to display the selected pacing location on a user interface.

In Example 15, the subject matter of any one or more of Examples 1-14 optionally includes the controller circuit that can be configured to generate a control signal to the electrostimulator to initiate or adjust electrostimulation being delivered to the selected pacing location to restore cardiac synchrony.

Example 16 is a method of pacing a heart to restore cardiac synchrony using a medical-device system, the method comprising: delivering electrostimulation respectively to two or more candidate locations at or near a cardiac conduction system of a patient using an electrostimulator; receiving respective heart sound information sensed from a patient in response to the delivery of the electrostimulation at or near the two or more candidate locations; generating heart sound metrics for the two or more candidate locations using the respective heart sound information; selecting at least one of the candidate locations as a pacing location for subsequent electrostimulation based at least in part on the generated heart sound metrics; and providing the selected pacing location to a user or to a process executable by the medical-device system.

In Example 17, the subject matter of Example 16 optionally includes selecting the pacing location for subsequent electrostimulation based on a comparison of the generated heart sound metrics for the two or more candidate locations.

In Example 18, the subject matter of any one or more of Examples 16-17 optionally includes generating a reference heart sound metric using heart sound information sensed from the patient without the electrostimulation being delivered to any of the two or more candidate locations, wherein that the selected pacing location corresponds to a difference between the generated heart sound metric in response to the electrostimulation and the reference heart sound metric exceeding a threshold value.

In Example 19, the subject matter of any one or more of Examples 16-18 optionally includes delivering the electrostimulation respectively to the two or more candidate locations that can include positioning a lead with associated one or more electrodes at or near two or more candidate locations in a His-bundle or septal region of the heart, and inserting a distal tip of the lead into the His-bundle or septal region, wherein the selected pacing location for subsequent electrostimulation includes one or more of a selected His-bundle or septal site, or a selected depth of insertion of the distal tip of the lead. In some examples, the two or more candidate locations in the His-bundle or septal region include two or more candidate locations at or near one or more of a left bundle branch or a right bundle branch of the heart.

In Example 20, the subject matter of any one or more of Examples 16-19 optionally includes the heart sound metric that can include at least one of: an S1 heart sound intensity; a pre-ejection period; a split S1 pattern; or a paradoxical split S2 pattern.

In Example 21, the subject matter of any one or more of Examples 16-20 optionally include: delivering electrostimulation at the selected pacing location in accordance with varying pacing dosage or timing parameter values; receiving respective secondary heart sound information sensed from a patient in response to the delivery of the electrostimulation in accordance with the varying pacing dosage or timing parameter values at the selected pacing location; generating secondary heart sound metrics using the respective secondary heart sound information; and determining a pacing dosage or timing parameter value based at least in part on the generated secondary heart sound metrics corresponding to the varying pacing dosage or timing parameter values.

In Example 22, the subject matter of any one or more of Examples 16-21 optionally includes displaying in real time the generated heart sound metrics on a user interface while the electrostimulation is being delivered respectively to the one or more candidate location.

The systems, devices, and methods discussed in this document may improve the technology of device-based cardiac pacing, particularly CSP, yet with little to no additional cost or system complexity. CSP may activate the His-Purkinje system, thereby preserving ventricular synchrony and improving cardiac performance without structural and functional impairment to the heart. CSP as discussed in the present document can leverage the electrophysiology of the His-Purkinje system, and improve pacing efficiency utilizing the natural conduction mechanisms of the heart, while reducing long-term harmful hemodynamic effects associated with conventional RV apical pacing used for HF management. Compared to conventional ECG-based pacing optimization techniques, the heart sound-based pacing optimization as described in the present document takes advantage of existing sensors (e.g., accelerometers) capable of measuring heart sounds. The heart sound metrics can be derived from the heart sound signal using less complicated techniques that require less computation time. The heart sound-based pacing site and pacing parameter optimization can increase the chance of capturing the cardiac conductive system and thus improve the overall success rate of CSP therapy, promote synchrony via patient's native His-Purkinje system, and produce favorable cardiac performance and hemodynamic outcome. With more effective and efficient pacing site optimization as described in this document, ineffective pacing (i.e., loss of capture) can be reduced, and battery power of an cardiac pacing system or device can be saved, and extend device longevity can be achieved without compromising effective CSP therapy. As such, the subject matter discussed in this document improves the functionality of an cardiac pacing system or device.

While His-bundle pacing is specifically discussed in various examples in this document, this is meant only by way of example and not limitation. It is within the contemplation of the inventors, and within the scope of this document, that the systems, devices, and methods described in accordance with various embodiments herein may be similarly applied to controllably stimulate candidate locations of the right or left bundle branches, fascicles, the Purkinje fibers, among other conductive tissue of the cardiac conduction system, and to determine an optimal or desired pacing location and optimal or desired pacing parameters.

This Overview provides some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates generally and example of a cardiac disease management system and portions of an environment in which the system may operate.

FIG. 3 illustrates generally an example of a heart sound-based cardiac pacing system for electrically stimulating a patient's natural cardiac conduction system.

FIG. 4 illustrates a portion of a user interface that displays S1 intensity in real time as the pacing shifts from one candidate location to another at or near the cardiac conduction system.

FIG. 5 illustrates generally an example of a method for pacing a location at or near a cardiac conduction system of the heart to restore cardiac synchrony

FIG. 6 illustrates generally a block diagram of an example machine upon which any one or more of the techniques (e.g., methodologies) discussed herein may perform.

### DETAILED DESCRIPTION

Disclosed herein are systems, devices, and methods for electrically stimulating a patient's natural cardiac conduction system (e.g., the His-Purkinje system) at selected pacing locations based on heart sounds. A medical-device system includes a data receiver to receive heart sound information sensed from a patient, an electrostimulation circuit to deliver electrostimulation to the patient's natural cardiac conduction system, and a controller circuit to generate, from the heart sound information sensed in response to the electrostimulation being delivered respectively to two or more candidate locations at or near the cardiac conduction system, heart sound metrics respectively for the two or more candidate locations. Based on the heart sound metrics, the controller circuit can select at least one of the candidate locations as a pacing location for subsequent electrostimulation. Electrostimulation can be delivered to the selected pacing location to restore cardiac synchrony.

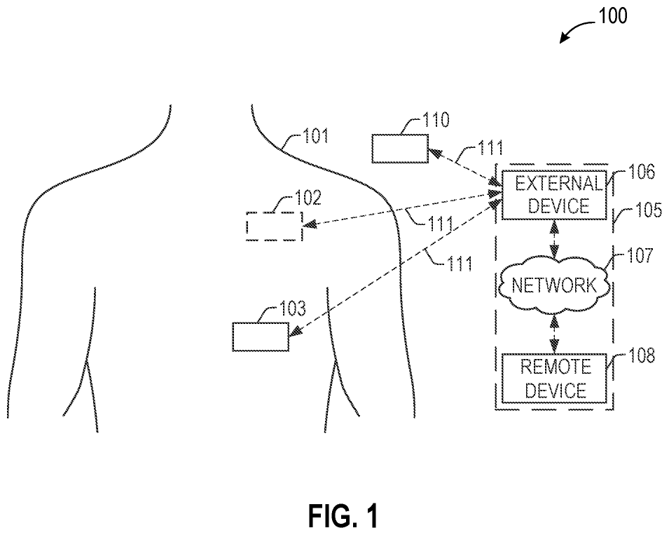

FIG. 1 illustrates generally an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 may include one or more ambulatory medical devices, an external system 105, and a communication link 111 providing for communication between the one or more ambulatory medical devices and the external system 105. The one or more ambulatory medical devices may include an implantable medical device (EVID) 102, a wearable medical device (WMD) 103, or one or more other implantable, leadless, subcutaneous, external, wearable, or ambulatory medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 may include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 may include a monitor implanted, for example, subcutaneously in the chest of patient 101, the IMD 102 including a housing containing circuitry and, in certain examples, one or more sensors, such as a temperature sensor, etc.

The IMD 102 may include an assessment circuit configured to detect or determine specific physiologic information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy may include delivery of one or more drugs to the patient 101, such as using the IMD 102 or one or more of the other ambulatory medical devices, etc. In some examples, therapy may include cardiac resynchronization therapy for rectifying dyssynchrony and improving cardiac function in heart failure patients. In other examples, the IMD 102 may include a drug delivery system, such as a drug infusion pump to deliver drugs to the patient for managing arrhythmias or complications from arrhythmias, hypertension, or one or more other physiologic conditions. In other examples, the IMD 102 may include one or more electrodes configured to stimulate the nervous system of the patient or to provide stimulation to the muscles of the patient airway, etc.

The WMD 103 may include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

In an example, the IMD 102 or the WMD 103 may include or be coupled to an implantable or wearable sensor to sense a heart sound signal, and include a heart sound recognition circuit to recognize one or more heart sound components such as S1, S2, S3, or S4 based on a spectral entropy time series derived from the sensed heart sound signal. Also included in the IMD 102 or the WMD 103 is a heart sound-based event detector circuit that can detect a physiologic event (e.g., a cardiac arrhythmia episode, or a worsening heart failure (WHF) event) based at least on a heart sound metric of the detected one or more heart sound component. Examples of such heart sound metric may include an amplitude, or timing of the heart sound component within a cardiac cycle relative to a fiducial point. In some examples, at least a portion of the heart sound recognition circuit and/or the heart sound-based event detector circuit may be implemented in and executed by the external system 105.

The external system 105 may include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This may include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information may include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 may include an external device 106 in proximity of the one or more ambulatory medical devices, and a remote device 108 in a location relatively distant from the one or more ambulatory medical devices, in communication with the external device 106 via a communication network 107. Examples of the external device 106 may include a medical device programmer. The remote device 108 can be configured to evaluate collected patient or patient information and provide alert notifications, among other possible functions. In an example, the remote device 108 may include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server may include a memory device to store the data in a patient database. The server may include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications may include a Web page update, phone or pager call, E-mail, SMS, text or “Instant” message, as well as a message to the patient and a simultaneous direct notification to emergency services and to the clinician. Other alert notifications are possible. The server may include an alert prioritizer circuit configured to prioritize the alert notifications. For example, an alert of a detected physiologic event can be prioritized using a similarity metric between the physiological data associated with the detected physiologic event to physiological data associated with the historical alerts.

The remote device 108 may additionally include one or more locally configured clients or remote clients securely connected over the communication network 107 to the server. Examples of the clients may include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiologic events to a system user, such as the patient or a clinician, or to a process including, for example, an instance of a computer program executable in a microprocessor. In an example, the process may include an automated generation of recommendations for anti-arrhythmic therapy, or a recommendation for further diagnostic test or treatment. In an example, the external device 106 or the remote device 108 may include a respective display unit for displaying the physiologic or functional signals, or alerts, alarms, emergency calls, or other forms of warnings to signal the detection of arrhythmias. In some examples, the external system 105 may include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” may include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 may include a local external implantable medical device programmer. The external system 105 may include a remote patient management system that can monitor patient status or adjust one or more therapies such as from a remote location.

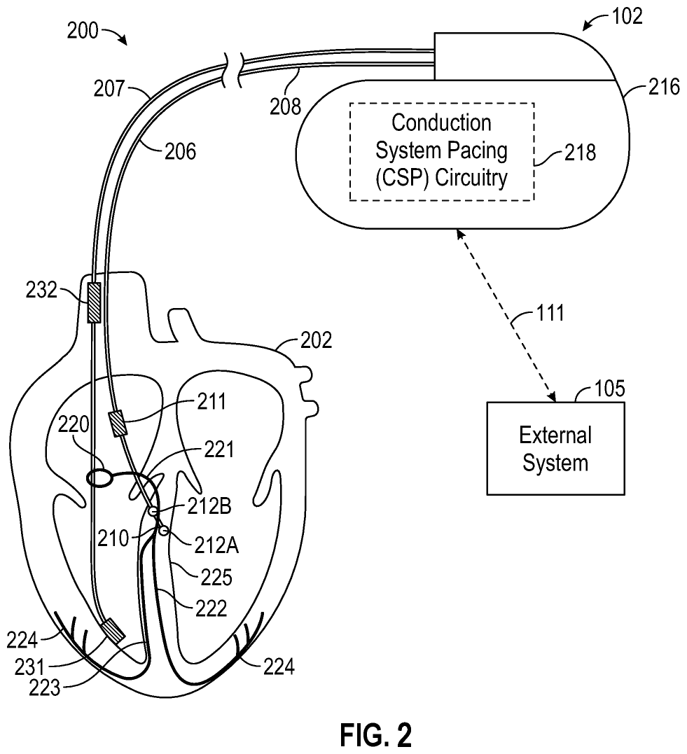

FIG. 2 is a schematic diagram illustrating an embodiment of a cardiac disease management system 200 and portions of an environment in which the system 200 may operate. The cardiac disease management system 200 may perform a range of activities, including remote patient monitoring, diagnosis of a disease condition, and providing a therapy to treat the disease condition and to improve patient outcome. In an example, the therapy may include conduction system pacing (CSP), such as His-bundle pacing (HBP), septa pacing such as left bundle branch pacing (LBBP) or right bundle branch pacing (RBBP), or pacing at other parts the His-Purkinje conduction system. One or more of these activities may be performed proximal to a patient (e.g., in the patient's home or office), through a centralized server (e.g., in a hospital, clinic or physician's office), or through a remote workstation (e.g., a secure mobile computing device).

The cardiac disease management system 200 may be coupled to a patient's heart 202. The cardiac disease management system 200 includes an ambulatory medical device (AMD), which can be an implantable device subcutaneously implanted in a chest, abdomen, or other parts of the patient, a subcutaneous monitor or diagnostic device, or a wearable medical device such as a patch-based device or a smart wearable or accessory, among others. In the example as illustrated in FIG. 2, the AMD can be an implantable medical device, such as the IMD 102 as illustrated in FIG. 1.

The cardiac disease management system 200 may include a lead system electrically coupled to the IMD 102. The lead system may include one or more transvenously, percutaneously, or minimally invasive placed leads or catheters. Each lead or catheter may include one or more electrodes. The arrangements and uses of the lead system and the associated electrodes may be determined by patient need and capability of the IMD 102. The associated electrodes on the lead system may be positioned at the patient's thorax or abdomen to sense a physiological signal indicative of cardiac activity, or a physiological response to stimulation of a target tissue. The lead system may be surgically inserted into, or positioned on the surface of, a heart 202. The electrodes associated with the lead system may be disposed in a target site in a right atrium (RA), a right ventricle (RV), a left atrium (LA), or a left ventricle (LV), or other body parts. Stimulation energy may be delivered to a target site via one or more of these electrodes. Some electrodes may be used for sensing cardiac activity, such as an intrinsic or evoked cardiac electrical activity.

In the illustrated example, the lead system may include a lead 206 having a proximal end 208 configured to be connected to the IMD 102, and a distal end 210 that includes one or more electrodes configured to deliver stimulation energy, such as in a form of pacing pulses, to the His bundle 221. FIG. 2 illustrates, by way of example and not limitation, two electrodes including a tip electrode 212A and a ring electrode 212B. Additional electrodes may be included in the lead 206 for sensing electrical activity or for delivering stimulation energy, such as an atrial lead 211. The lead 206 may be placed such that one or more electrodes, such as electrodes 212A-212B, are positioned in or near a His bundle 221, a region distal to the blocked or slowly conducting AV node, an interventricular septum region, or a right atrial region near the His bundle 221. Alternatively, one or more of the electrodes 212A-212B, or other electrodes on the lead 206, can be positioned at the interventricular septum 225 to provide septal pacing, which may include one or more of left bundle branch (LBB) pacing or right bundle branch (RBB) pacing. As part of the natural electrical conduction system of the heart 202, the His bundle 221 transmits the electrical impulses from the AV node 220 to the point of the apex of the fascicular branches via the left bundle branch 222 and the right bundle branch 223 within the interventricular septum 225. The left bundle branch 222 and the right bundle branch 223 leads to the Purkinje fibers 224, which provide electrical conduction to the ventricles, causing the ventricles to contract. In some examples, the lead 206 may be placed such that one or more electrodes associated with the lead 206, such as electrodes 212A-212B, are positioned at or near other parts of the natural cardiac conduction system, such as one of the bundle branches 222 or 223, the Purkinje fibers 224, or other conductive tissue, in addition to or in lieu of a region at or near the His bundle 221. In some example, the distal tip of the lead 206 may be inserted into the His bundle or a septal region at a desired depth, such that one or more of the electrodes 212A-212B are in closer proximity to the cardiac conductive tissue of the His-Purkinje system, thereby increasing the success rate of capturing the cardiac conductive tissue via electrical stimulation.

In some examples, one or more leads other than the lead 206 may additionally or alternatively be used to provide CSP (e.g., HBP, septal pacing such as LBBP or RBBP, among others) via one or more electrodes associated therewith. Such leads may include transvenous, subcutaneous, endocardial, epicardial, or non-invasive leads. For example, a ventricular lead 207 may include electrodes configured to be positioned at the septum 225, or optionally inserted into a septal region at a desired depth, to provide septal pacing such as LBBP or RBBP therein. The ventricular lead 207 may additionally include one or more electrodes, such as a distal electrode 231 and a proximal electrode 232, for cardiac sensing and/or pacing the ventricle.

In an example, the lead 206 may be a single pass lead having a plurality electrodes for stimulating multiple cardiac sites, including electrodes disposed at or near the His bundle (e.g., the electrodes 212A-212B) and electrodes disposed in one or more of RA, RV, LA, or LV of the heart 202. In some examples, in addition to the lead 206, the lead system may include separate leads for placement in different heart chambers or sites, such as an RA lead having one or more RA electrodes to stimulate a portion of RA or to sense RA electrical activity, a RV lead having one or more RV electrodes to stimulate a portion of RV or to sense RV electrical activity, or an LV lead having one or more LV electrodes to stimulate a portion of LV or to sense LV activity. In some examples, the cardiac disease management system 200 may include one or more leadless stimulators/sensors untethered to a lead and in wireless communication with the IMD 102. The leadless stimulators/sensors may deliver electrostimulation, sense a physiological signal, such as cardiac electrical signals in response to cardiac stimulation, and transmit the sensed data to the IMD 102.

The IMD 102 may include a hermetically sealed housing 216 that houses one or more of an electrostimulation circuit, a sensing circuit, a control circuit, a communication circuit, and a battery, among other components. In an example, the IMD 102 includes cardiac conduction system pacing (CSP) circuitry 218 configured to provide electrostimulation to a location at or near the patient's cardiac conduction system (e.g., the His-Purkinje system), such as the His bundle 221, the bundle branches 222 or 223, the Purkinje fibers 224, or other conductive tissue of the cardiac conduction system via the lead 206 and the associated electrodes 212A or 212B. The CSP circuitry 218 may be programmed to deliver unipolar His-bundle pacing, where the pacing energy (current or voltage) is applied between one of the electrodes 212A-212B (e.g., as a cathode) and the housing 216 (e.g., as an anode). Alternatively, the CSP circuitry 218 may be programmed to deliver bipolar His-bundle pacing, where the pacing energy (current or voltage) is applied between two electrodes positioned at or near the His bundle, such as between the electrodes 212A and 212B. In some examples, electrodes used for unipolar or multipolar (e.g., bipolar or quadripolar) His-bundle pacing may be selected by a system user from a plurality of candidate electrodes from a given lead or multiple separate leads comprising the pacing system, and programmed into the CSP circuitry 218. In some examples, HBP pulses may be provide by a leadless device, such as a leadless cardiac pacemakers (LCP). One or more electrodes may be distributed on the body of the LCP and in contact with His-bundle region to deliver pacing pulses thereto.

The CSP circuitry 218 may include sensing circuitry to sense a physiological signal using one or more electrodes associated with the lead system or a physiological sensor. Examples of the physiological signal may include an electrocardiogram (ECG), an intracardiac electrogram (EGM) such as an atrial EGM, a ventricular EGM, or a His bundle EGM, a heart rate or a pulse rate signal, a thoracic impedance signal, a cardiac impedance signal, an arterial pressure signal, a pulmonary artery pressure signal, a left atrial pressure signal, an RV pressure signal, an LV coronary pressure signal, a coronary blood temperature signal, a blood oxygen saturation signal, a cardiac acceleration signal, a heart sound signal, an intracardiac acceleration signal, a respiration signal, or a physical activity or exertion level signal, a cardiac timing signal, among others. In an example, the sensing circuitry of the CSP circuitry 218 may be coupled to a heart sound sensor to sense heart sound information. The heart sound information may be sensed in response to electrostimulation being delivered respectively to two or more candidate locations at or near the His-Purkinje system, such as one or more His-bundle locations, one or more left or right bundle branch locations, one or more septal locations (e.g., locations at or near a left bundle branch or a right bundle branch), among other candidate locations of the His-Purkinje conduction system. The CSP circuitry 218 may generate heart sound metrics for each of the two or more candidate locations at or near the His-Purkinje system using the corresponding heart sound information. The CSP circuitry 218 can determine a desired pacing location at or near the His-Purkinje system based on the generated heart sound metrics, and deliver electrostimulation (e.g., cardiac pacing) at the determined desired pacing location to restore cardiac synchrony or to improve cardiac performance. In some examples, in addition to determining the desired pacing location, the CSP circuitry 218 may further determine a desired pacing dosage or timing parameter value based on the heart sound metrics derived from heart sound information sensed in response to the electrostimulation being delivered at the desired pacing location in accordance with varying pacing dosage or timing parameter values. In some examples, the CSP circuitry 218 may provide ventricular backup pacing if the CSP does not capture the conductive tissue at the pacing site and fail to adequately activate the ventricles and restore cardiac synchrony.

The IMD 102 may communicate with the external system 105 via a communication link 111. The external system 105, as described above with respect to FIG. 1, may include a dedicated hardware/software system such as a programmer, a remote server-based patient management system, or alternatively a system defined predominantly by software running on a standard personal computer. The external system 105 may include a proximal external device such as a programmer device in proximity of the IMD 102. A clinician may manage the patient 101 through the IMD 102 via the communication link 111, including, among other operations, initiating or adjusting a therapy such as CSP. Additionally, the external system 105 may receive device data from the IMD 102 via the communication link 111. Examples of the device data may include real-time or stored physiological signals collected from the patient 101, physiological response to therapies delivered to the patient 101, or device operational status of the IMD 102 (e.g., battery status and lead impedance).

The external system 105 may monitor patient condition and the function of IMD 102. In various embodiments, the external system 105 may include a user interface to display received information to the user, and receive user input for operation control of the IMD 102. In an example, the external system 105 can be configured to verify pacing capture status, perform pacing threshold test to determine an HBP threshold. The capture verification and threshold testing may be executed periodically, or triggered by a specific event such as a user command. A user may use the external system 105 to program the IMD 102, such as to configure a pacing vector (e.g., specifying anode and cathode electrodes) to deliver HBP, or to configure a sense vector to sense a physiological signal.

In the example as shown in FIG. 2, the CSP circuitry 218 is completely within the IMD 102. In some examples, a portion or the entirety of the CSP circuitry 218 may be included in another device separate from the IMD 102, such as an external device. Examples of such external device can include the WMD 103, a portable pacing system analyzer (PSA) device, a programmer device for programming the IMP 102, among other electrostimulator devices. The external device can be coupled to one or more leads, such as the lead 206 or the lead 207, to provide electrostimulation respectively at one or more candidate pacing locations at or near the cardiac conduction system. The external device can receive heart sound information sensed from the patient in response to the electrostimulation at respective candidate locations, and determine a desired pacing location based at least on the heart sound information.

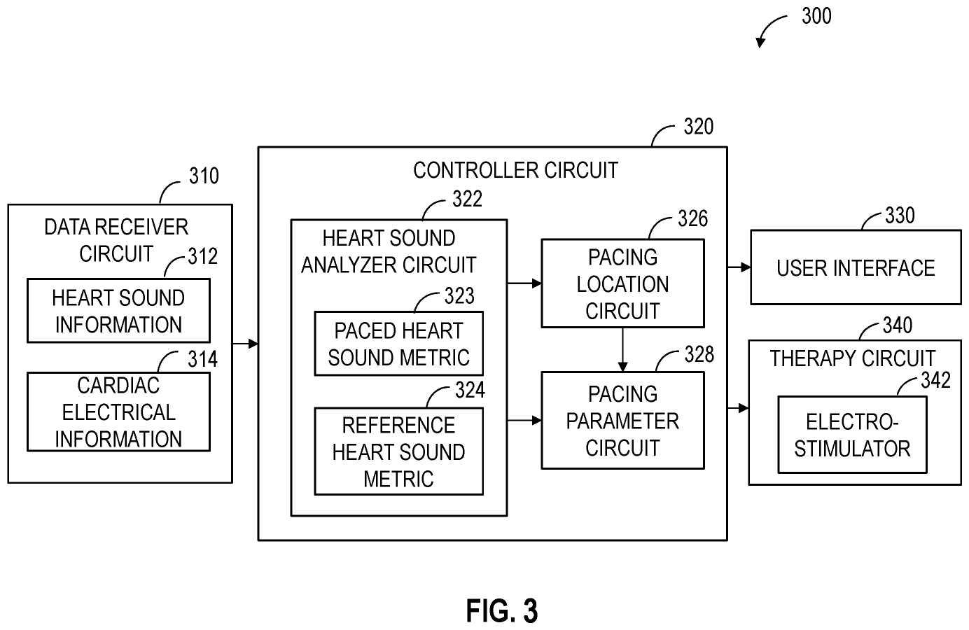

FIG. 3 illustrates generally an example of a heart sound-based cardiac pacing system 300 for providing electrostimulation to a patient's natural cardiac conduction system (e.g., the His-Purkinje system). The heart sound-based cardiac pacing system 300 can determine an optimal or desired pacing location at or near the cardiac conduction system, and optionally optimal or desired pacing parameters, to more effectively capture the cardiac conductive tissue, restore synchrony, and improve the patient's cardiac performance. The heart sound-based cardiac pacing system 300 may include one or more of a data receiver circuit 310, a controller circuit 320, a user interface 330, and a therapy circuit 340. At least a portion of the heart sound-based cardiac pacing system 300 may be implemented in the IMP 102, the WMD 103, or the external system 105 such as one or more of the external device 106 or the remote device 108.

The data receiver circuit 310 may receive physiologic information from a patient. In an example, the data receiver circuit 310 may include a sense amplifier circuit configured to sense a physiologic signal from a patient via a physiologic sensor, such as an implantable, wearable, or otherwise ambulatory sensor or electrodes associated with the patient. The sensor may be incorporated into, or otherwise associated with an ambulatory device such as the IMD 102 or the WMD 103. In some examples, the physiologic signals sensed from a patient may be stored in a storage device, such as an electronic medical record (EMR) system. The data receiver circuit 310 may receive the physiologic signal from the storage device, such as in response to a user command or a triggering event. By way of example and not limitation and as illustrated in FIG. 3, the data receiver circuit 310 may receive heart sound information 312 and cardiac electrical information 314. The heart sound information, which can be sensed using a heart sound sensor from the patient, includes one or more of S1, S2, S3, or S4 heart sound components. In an example, the heart sound information 312 may include a body motion/vibration signal indicative of cardiac vibration, which is correlated to or indicative of heart sounds. In an example, at least one heart sound sensor can be included in the heart sound-based cardiac pacing system 300. Examples of the heart sound sensor may include an accelerometer, an acoustic sensor, a microphone, a piezo-based sensor, or other vibrational or acoustic sensors. The accelerometer can be a one-axis, a two-axis, or a three-axis accelerometer. Examples of the accelerometer may include flexible piezoelectric crystal (e.g., quartz) accelerometer or capacitive accelerometer, fabricated using micro electro-mechanical systems (MEMS) technology. The heart sound sensor may be included in the IMD 102 or the WMD 103, or disposed on a lead such as a part of the lead system associated with the IMD 102 or the WMD 103. In an example, an accelerometer (or other sensors) may sense an epicardial or endocardial acceleration (EA) signal from a portion of a heart, such as on an endocardial or epicardial surface of one of a left ventricle, a right ventricle, a left atrium, or a right atrium. The EA signal may contain components corresponding to various heart sound components such as one or more of S1, S2, S3, or S4 components.

The cardiac electrical information 314 may include, for example, surface electrocardiogram (ECG) sensed from electrodes placed on the body surface, subcutaneous ECG sensed from electrodes placed under the skin, intracardiac electrogram (EGM) sensed from the one or more implantable electrodes. The cardiac electrical information 314 may be sensed via electrodes included in or communicatively coupled to the IMD 102 or the WMD 103. The cardiac electrical information 314 may additionally include heart rate, heart rate variability, cardiac timing parameters, etc., which can be measured or determined from the cardiac electrical information 314.

In some examples, the data receiver circuit 310 may receive other physiological or functional information of the patient including, for example, physical activity signal, posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide (BNP), blood panel, sodium and potassium levels, glucose level and other biomarkers and bio-chemical markers, among others.

The controller circuit 320 can determine an optimal or desired pacing location at or near the cardiac conduction system, and optionally optimal or desired pacing parameters for delivering electrostimulation to the patient's cardiac conduction system. The controller circuit 320 can be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The controller circuit 320 may include circuit sets comprising one or more other circuits or sub-circuits, such as a heart sound analyzer circuit 322, a pacing location circuit 326, and a pacing parameter circuit 328. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The heart sound analyzer circuit 322 can preprocess the heart sound information 312, such as using a filter or a filter bank to remove or attenuate one or more of low-frequency signal baseline drift, high frequency noise, or other unwanted frequency contents. In an example, the heart sound segment may be band-pass filtered to a frequency range of approximately 5-90 Hz, or approximately 9-90 Hz. In an example, the filter may include a double or higher-order differentiator configured to calculate a double or higher-order differentiation of the heart sound signal. The heart sound analyzer circuit 322 may detect one or more heart sound components (e.g., S1, S2, S3, or S4) from the preprocessed heart sound signal. The heart sound component can be recognized based on heart sound signal amplitude, power, timing, morphology, spectrum, statistical analysis, or signal complexity analysis (e.g., signal entropy). In some examples, the heart sound analyzer circuit 322 can detect the heart sound components further using the cardiac electrical information 314, such as timings of QRS complex or R wave within a cardiac cycle, heart rates, heart rate variability, etc.

The heart sound analyzer circuit 322 can generate a heart sound metric from the received heart sound information 312. In some examples, the heart sound information 312 (optionally along with other information received by the data receiver circuit 310), may be sensed in response to electrostimulation being delivered respectively to two or more candidate locations at or near the patient's cardiac conduction system, such as one or more His-bundle locations, one or more left or right bundle branch locations, one or more septal locations, among other candidate locations of the His-Purkinje conduction system. The electrostimulation may be delivered by an electrostimulator 342, which can be a part of the therapy circuit 340. In an example, the electromotor 342 can be included in the IMD 102 or the WMD 103. In another example, the electromotor 342 can be an external device separate from the IMD 102 or the WMD 103, such as being within the external device 106 (e.g., a programmer for programming the IMD 102 or the WMD 103) or a portable pacing system analyzer (PSA) device. The electrostimulator 342 can be electrically coupled to at least one electrode positionable at the two or more candidate locations to deliver the electrostimulation therein. The at least one electrode can be associated with a lead, such as the electrodes 212A and 212B associated with the lead 206 as shown in FIG. 2. The lead has a proximal end coupled to the electrostimulator 342 and a distal tip configured to be inserted into a His-bundle or septal site. The two or more candidate locations at or near the His-Purkinje system may include candidate His-bundle or septal pacing sites (e.g., LBBP sites or RBBP sites), and variable (candidate) depths of insertion of the distal tip of the lead into the His bundle or a septal region. Heart sound information may be sensed during the electrostimulation at a candidate site or immediately after the electrostimulation within a specific time period to account for heart sound response delay following the cessation of electrostimulation. Such heart sound information is hereinafter referred to as paced heart sound information. In an example where multiple candidate locations {P1, P2, . . . , Pn} at or near the cardiac conduction system are stimulated, multiple paced heart sound signals {HS1, HS2, . . . HSn} can be sensed each corresponding to the electrostimulation at respective candidate locations. Each candidate location corresponds to a His-bundle or septal site and a insertion depth of the distal tip of the lead at the His bundle or a septal site.

The heart sound analyzer circuit 322 can generate, for each of the one or more candidate pacing locations (e.g., candidate site Pi), a paced heart sound metric 323 (pXi) using the paced heart sound information (HSi) for that corresponding candidate site. The paced heart sound metrics can be correlated to the level of cardiac synchrony and indicative of cardiac performance. The paced heart sound metric 323 can be generated based on one or more of the heart sound components (e.g., S1, S2, S3, or S4) detected from the paced heart sound signal (e.g., HSi), and optionally further based on the cardiac electrical information 314 acquired during pacing. In an example where multiple candidate locations {P1, P2, . . . , Pn} are tested, multiple paced heart sound metrics {pX1, pX2, . . . , pXn} can be generated respectively from the heart sound information {HS1, HS2, . . . , HSn} each corresponding to the electrostimulation at respective candidate locations.

In one example, the paced heart sound metric 323 can include an intensity (e.g., signal amplitude or power) of a heart sound component, such as S1 intensity. S1 is associated with the vibrations produced by the heart during closure of the atrioventricular (mitral and tricuspid) valves as the ventricular pressures exceed atrial pressures at the beginning of systole. Cardiac dyssynchrony, such as due to left bundle branch block (LBBB), can significantly reduce S1 intensity. Electrostimulation at a proper location at or near the cardiac conduction system can restore cardiac synchrony and boost contractility, as reflected by an increased S1 intensity.

In another example, the paced heart sound metric 323 can include a cardiac timing interval. Examples of the cardiac timing interval can include a pre-ejection period (PEP), which represents a latency between the onset of the QRS to the S1 heart sound, or Q-S1 interval. Cardiac dyssynchrony, such as due to LBBB, can significantly lengthen the Q-S1 interval. Electrostimulation at a proper location at or near the cardiac conduction system can restore cardiac synchrony and boost contractility, as reflected by a shortening of Q-S1 interval. Other cardiac timing intervals may also be included in the paced heart sound metric 323, including, for example, a systolic timing interval (STI) representing a time interval from the onset of the QRS complex on the ECG to the S2 heart sound, a left-ventricular ejection time (LVET) representing a time interval between S1 and S2 heart sounds, or a diastolic timing interval (DTI) representing a time interval between the S2 heart sound and the onset of the subsequent QRS complex on the ECG, among others. These heart sound-based cardiac timing intervals may be correlated with cardiac contractility or cardiac diastolic function of the heart. The paced heart sound metric 323 may further include PEP/LVET ratio, STI/DTI ratio, STI/cycle length (CL) ratio, or DTI/CL ratio, or other composite metrics.

In another example, the paced heart sound metric 323 can include a split S1 pattern. A split S1 occurs when the mitral valve closes significantly before the tricuspid valve, allowing each valve to make a separate sound or vibrations detectable by a heart sound sensor. Although S1 splitting is normal in many patients, it is rarely seen in heart failure patients with LBBB. Electrostimulation at a proper location at or near the cardiac conduction system can restore cardiac synchrony and boost contractility, which can promote reappearance of split S1 in those heart failure patients. The heart sound analyzer circuit 322 can detect the presence or absence of split S1 pattern based on signal morphology of the paced heart sound signal. For example, if more than one dominant S1 peak is detected in a S1 detection window, a detection of split S1 is declared. Alternatively, the split S1 pattern can be detected if the width of S1 energy peak (e.g., measured at 75% of the S1 energy peak value) within the S1 detection window exceeds a specific threshold. The S1 window can begin at a predetermined offset from the QRS complex or R wave of an ECG and have a predetermined window size (duration). The heart sound analyzer circuit 322 may additionally calculate an occurrence rate of split S1 pattern during a specific monitoring time period or a specific number of cardiac cycles.

In yet another example, the paced heart sound metric 323 can include a paradoxical split S2 pattern. A paradoxical split S2 pattern is characterized by a presence of split S2 during the expiration and an absence of split S2 during inspiration. A paradoxical split S2 occurs in any setting that delays the closure of the aortic valve, such as LBBB. Electrostimulation at a proper location at or near the cardiac conduction system can restore cardiac synchrony and correct the paradoxical split S2, as reflected by the absence of split S2 pattern during both the inspiration and expiration phase. The data receiver circuit 310 may receive respiration information including an inspiration phase and an expiration phase in a respiration cycle. The heart sound analyzer circuit 322 can detect the presence or absence of paradoxical split S2 pattern based on signal morphologies of a first portion of the paced heart sound signal during the inspiration phase and signal morphologies of a second portion of the paced heart sound signal during the expiration phase. A paradoxical split S2 pattern is declared if split S2 is detected only during the expiration phase but not during the inspiration phase. The heart sound analyzer circuit 322 may additionally calculate an occurrence rate of paradoxical split S2 pattern during a specific monitoring time period or a specific number of cardiac cycles.

In some examples, the received heart sound information 312 may additionally include heart sounds sensed from the patient without CSP being delivered to the heart, such as during an intrinsic cardiac rhythm. Similarly, the cardiac electrical information 314 may include cardiac electrical information sensed without the CSP being delivered to the heart. Such heart sound information is hereinafter referred to as baseline heart sound information. The heart sound analyzer circuit 322 can generate a reference heart sound metric 324 (rX) using the baseline heart sound information. Similar to various examples of the paced heart sound metric 323 (pX), the reference heart sound metric 324 may include a reference intensity of heart sound component such as a reference S1 intensity, a reference cardiac timing interval such as a reference PEP or reference Q-S1 interval, a reference detection or occurrence rate of split S1 pattern, or a reference detection or a reference occurrence rate of paradoxical split S2 pattern, among others.

The pacing location circuit 326 can determine an optimal or desired pacing location (P*) at or near the cardiac conduction system based at least in part on the paced heart sound metrics 323 respectively generated for the two or more candidate locations. In an example, the optimal or desired pacing location (P*) can be selected from the two or more candidate locations, such as from the multiple candidate locations {P1, P2, . . . , Pn}. A candidate location Pi with the corresponding paced heart sound metric pXi satisfying a specific condition can be determined to be the optimal or desired pacing location (P*). For example, the optimal or desired pacing location P* can be determined to be a candidate location with the corresponding S1 intensity exceeding a threshold; the corresponding pre-ejection period (Q-S1 interval) falling below a threshold; a corresponding detection of a presence of split S1 pattern or an occurrence rate of split S1 pattern exceeding a threshold; or a corresponding non-detection (i.e. absence) of the paradoxical split S2 pattern or an occurrence rate of the paradoxical split S2 pattern falling below a threshold.

In some examples, the optimal or desired pacing location (P*) can be selected from the multiple candidate locations {P1, P2, . . . , Pn} being tested based on a comparison of the paced heart sound metrics {pX1, pX2, . . . , pXn}. For example, the optimal or desired pacing location P* can be determined to be the candidate location that corresponds to the largest S1 intensity among all the candidate locations tested; the shortest pre-ejection period (Q-S1 interval) among all the candidate locations tested; the highest occurrence rate of split S1 pattern among all the candidate locations tested; or the lowest occurrence rate of the paradoxical split S2 pattern among all the candidate locations tested.

In some examples, the optimal or desired pacing location P* can be determined based at least in part on a comparison of the paced heart sound metrics 323 to the reference heart sound metric 324. For example, the pacing location circuit 326 can calculate a difference between the paced heart sound metric pXi at the candidate location Pi and the reference heart sound metric rX, and recognize Pi as the optimal or desired pacing location P* if the difference (pXi−rX) satisfies a specific condition. For example, the candidate location Pi can be determined as the optimal or desired pacing location P* if the corresponding S1 intensity difference exceeds a threshold amount, or if the corresponding Q-S1 interval is shorter than the reference Q-S1 interval and the difference between the two exceeds a threshold amount, or if a split S1 occurrence rate exceeds a reference split S1 occurrence rate by at least a threshold amount, or if a paradoxical split S2 occurrence rate falls below a reference paradoxical split S2 occurrence rate by at least a threshold amount.

In some examples, the pacing location circuit 326 may determine an optimal or desired pacing location (P*) using a combination of two or more different heart sound metrics. For example, a first heart sound metric (X) is used to determine a preliminary optimal or desired pacing location or a selected subset of the potential optimal or desired pacing locations. A second heart sound metric (Y), different from the first heart sound metric (X), can then be used to confirm the preliminary optimal or desired pacing location, or to determine the optimal or desired pacing location (P*) from the subset of the potential optimal or desired pacing locations determined based on the first heart sound metric X. The first and second heart sound metrics can be selected from, for example, a heart sound component intensity such as S1 intensity, a cardiac timing interval such as the pre-ejection period (Q-S1 interval), a split S1 pattern, or a paradoxical split S2 pattern, as described above. In an example, the paradoxical split S2 pattern can be used to confirm the preliminary optimal or desired pacing location, or to determine the optimal or desired pacing location (P*) from the selected subset of the potential optimal or desired pacing locations

In some examples, the pacing location circuit 326 may determine an optimal or desired pacing location (P*) using a combination of a heart sound metric and a non-heart sound metric. For example, a preliminary optimal or desired pacing location or a selected subset of the potential optimal or desired pacing locations can be first determined using a heart sound metric (such as the S1 intensity, a cardiac timing interval such as the Q-S1 interval, the split S1 pattern, or the paradoxical split S2 pattern as described above). A cardiac electrical signal metric (e.g., a QRS width) can then be used to confirm the preliminary optimal or desired pacing location, or to determine the optimal or desired pacing location (P*) from the selected subset of the potential optimal or desired pacing locations. Alternatively, a cardiac electrical signal metric (e.g., a QRS width) can be used to determine a preliminary optimal or desired pacing location or a selected subset of the potential optimal or desired pacing locations, and a heart sound metric can then be used to confirm the preliminary optimal or desired pacing location, or to determine the optimal or desired pacing location (P*) from the selected subset of the potential optimal or desired pacing locations.

The pacing parameter circuit 328 can determine an optimal or desired value of a pacing dosage or timing parameter using the heart sound metrics produced by the heart sound analyzer circuit 322. Examples of the pacing dosage or timing parameter can include pulse amplitude, pulse width, frequency, duration(s), total charge injected per unit time, cycling (e.g., on/off time), waveform shapes, spatial locations of waveform shapes, pulse shapes, number of phases, phase order, interphase time, charge balance, among others. In some examples, the optimal or desired pacing dosage and timing parameter can be determined for pacing at the optimal or desired pacing location P* that has been determined by the pacing location circuit 326. To determine the optimal or desired pacing parameter value, the pacing parameter circuit 328 can generate and track changes of the paced heart sound metric 323 at the optimal or desired pacing location P* in accordance with varying pacing dosage or timing parameter values. An optimal or desired pacing parameter value can be determined when the heart sound metric, corresponding to the varying pacing dosage or timing parameter values, satisfies a specific condition, such as exceeding a threshold or falling within a predetermine value range.

The user interface 330 may include an input unit and an output unit. In an example, at least a portion of the user interface 330 may be implemented in the external system 105. The input unit may receive user input for programming the data receiver circuit 310 and the controller circuit 320, such as parameters for sensing the heart sound signal, detecting a heart sound component, computing a heart sound metric, etc. The input unit may include a keyboard, on-screen keyboard, mouse, trackball, touchpad, touch-screen, or other pointing or navigating devices. The output unit may include a display for displaying the sensed heart sound signal, candidate pacing locations (including pacing sites and depth of insertion of lead tip into a His bundle or a septal site) and paced heart sound metrics corresponding to respective candidate pacing locations, the determined optimal or desired pacing location, and the determined optimal or desired pacing parameter value, among others.

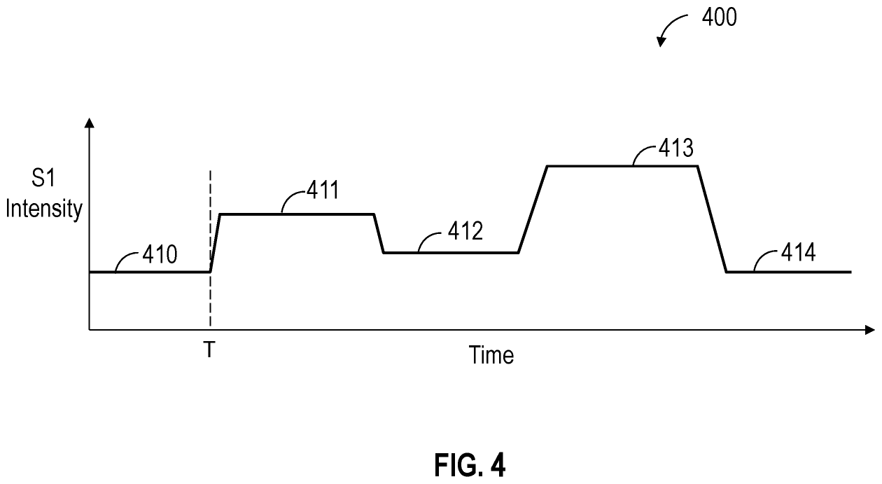

In an example, the controller circuit 320 can generate a control signal to the user interface 330 to display in real-time the heart sound metrics while the electrostimulation is being delivered at various candidate locations. Referring to FIG. 4, the diagram 400 illustrate a portion of user interface 330 that displays S1 intensity in real time as the pacing shifts from one candidate location to another at or near the cardiac conduction system. The real-time S1 intensity, which can be measured by the heart sound analyzer circuit 322, maintains a baseline level 410 before the pacing starts. At time T, pacing starts to be delivered at a first candidate location P1, resulting a S1 intensity level 411. Then the pacing is shifted to a second candidate location P2, resulting in a decreased S1 intensity level 412. The pacing is then moved to a third candidate location P3, which produces a substantial increase in S1 intensity level 413. Pacing at the fourth candidate location P4 produces the lowest S1 intensity level 414. As the S1 intensity level 413 is the largest among the tested candidate locations, the heart sound analyzer circuit 322 may select the corresponding candidate pacing site P3 as the optimal or desired location P* for use in subsequent cardiac pacing therapy.

Referring back to FIG. 3, the output unit of the user interface 330 may also present to a user, such as via a display unit, recommended therapy, including a change of parameters in the therapy provided by an implanted device, the prescription to get a device implanted, the initiation or change in a drug therapy, or other treatment options of a patient. The output unit may include a printer for printing hard copies of information that may be displayed on a display unit. The signals and information may be presented in a table, a chart, a diagram, or any other types of textual, tabular, or graphical presentation formats. The presentation of the output information may include audio or other media format. In an example, the output unit may generate alerts, alarms, emergency calls, or other forms of warnings to signal the system user about the detected medical events.

The therapy circuit 340 may be configured to deliver a therapy to the patient, such as in response to the detected physiologic event. The therapy may be preventive or therapeutic in nature such as to modify, restore, or improve patient neural, cardiac, or respiratory functions. Examples of the therapy may include electrostimulation therapy delivered to the heart, a nerve tissue, other target tissues, a cardioversion therapy, a defibrillation therapy, or drug therapy including delivering drug to the patient. The therapy circuit 340 may include the electrostimulator 342 to provide electrostimulation to two or more candidate locations at or near the patient's cardiac conduction system. In some examples, the therapy circuit 340 can modify an existing therapy, such as adjust a stimulation parameter or drug dosage. For example, electrostimulation may be delivered to the optimal or desired pacing location P* in accordance with the optimal or desired pacing dosage or timing parameter value.

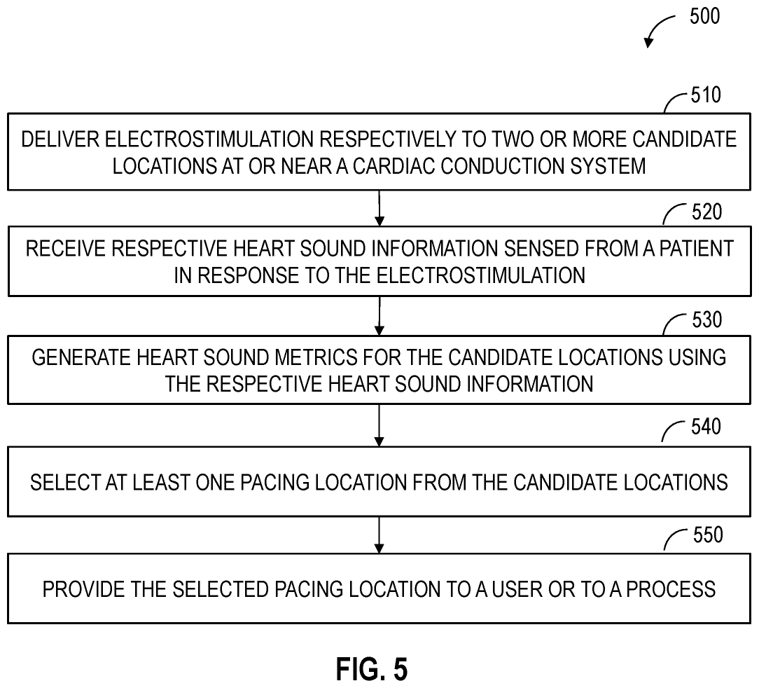

FIG. 5 illustrates generally an example of a method 500 for pacing a location at or near a cardiac conduction system of the heart to restore cardiac synchrony. The cardiac conduction system includes the His-Purkinje system comprising His bundle, left or right bundle branches, fascicles, or Purkinje fibers, among other conductive cardiac tissue. The method 500 may be implemented and executed in an ambulatory medical device such as an implantable or wearable medical device, or in a remote patient management system. In an example, the method 500 may be implemented in and executed by the IMD 102 or the WMD 103, the external system 105, or the heart sound-based heart sound-based cardiac pacing system 300.

The method 500 begins at 510 to deliver electrostimulation respectively to two or more candidate locations at or near a cardiac conduction system of a patient using an electrostimulator. Examples of the candidate locations can include one or more His-bundle locations, one or more left or right bundle branch locations, one or more septal locations, among other candidate locations of the His-Purkinje conduction system. The electrostimulator that provided the electrostimulation energy may be included in an ambulatory device such as the IMD 102 or the WMD 103, or alternatively an external device separate from the IMD 102 or the WMD 103, such as the external device 106 or a portable pacing system analyzer (PSA) device. The electrostimulator can be electrically coupled at least one electrode positionable at the two or more candidate locations to deliver the electrostimulation therein. The at least one electrode can be associated with a lead having a distal tip that can be inserted into the His-bundle or septal region of the heart. The candidate locations at or near the cardiac conduction system may include candidate His-bundle or septal pacing (e.g., LBBP or RBBP) sites, and variable (candidate) depths of insertion of the distal tip of the lead into the His bundle or a septal region.

At 520, heart sound information may be sensed from the patient in response to the delivery of the electrostimulation at or near the two or more candidate locations, such as during the electrostimulation being delivered to a candidate location, or immediately after the electrostimulation within a specific time period to account for heart sound response delay following the cessation of electrostimulation. The heart sounds may be detected using a sensor associated with or included in an ambulatory or wearable device. In some examples, endocardial acceleration signals sensed from inside the heart may be used to analyze heart sounds. Such heart sound information is also referred to as paced heart sound information, to distinguish from reference or baseline heart sound information sensed when no electrostimulation is delivered to any of the candidate locations. In an example where multiple candidate locations at or near the His-Purkinje system are stimulated, multiple paced heart sound signals can be sensed each corresponding to the electrostimulation at respective candidate locations.

At 530, heart sound metrics can be generated for each of the two or more candidate locations using the respective paced heart sound information. Such heart sound metrics, also referred to as paced heart sound metrics, can be based on one or more of the heart sound components (e.g., S1, S2, S3, or S4) detected from a paced heart sound signal, and optional further based on the cardiac electrical information or other physiological signals acquired during pacing. The paced heart sound metrics can be correlated to the level of cardiac synchrony and indicative of cardiac performance. By way of example and not limitation, the paced heart sound metrics may include an intensity (e.g., signal amplitude or power) of a heart sound component such as S1 intensity, a cardiac timing interval such as a pre-ejection period (PEP) or Q-S1 interval, a detection of or an occurrence rate of split S1 pattern, or a detection of or an occurrence rate of paradoxical split S2 pattern, as described above with reference to the paced heart sound metric 323 in FIG. 3.

In addition to the paced heart sound metrics generated from the paced heart sound information, in some examples, reference or baseline heart sound information may be sensed from the patient when no electrostimulation is delivered to any of the candidate locations, and a reference heart sound metric can be generated using the baseline heart sound information. Corresponding to various examples of the paced heart sound metric as described above, the reference heart sound metric may include a reference intensity of heart sound component such as a reference S1 intensity, a reference cardiac timing interval such as a reference PEP or reference Q-S1 interval, a reference detection or occurrence rate of split S1 pattern, or a reference detection or a reference occurrence rate of paradoxical split S2 pattern, among others.

At 540, an optimal or desired pacing location at or near the cardiac conduction system may be determined based at least in part on the paced heart sound metrics respectively generated for the two or more candidate locations. In an example, the optimal or desired pacing location (P*) can be selected from the two or more candidate locations, such as a candidate location with the corresponding S1 intensity exceeding a threshold; the corresponding pre-ejection period (Q-S1 interval) falling below a threshold; a corresponding detection of a presence of split S1 pattern or an occurrence rate of split S1 pattern exceeding a threshold; or a corresponding non-detection (i.e. absence) of the paradoxical split S2 pattern or an occurrence rate of the paradoxical split S2 pattern falling below a threshold. In another example, the optimal or desired pacing location (P*) can be selected from the multiple candidate locations being tested based on a comparison of the corresponding paced heart sound metrics, such as a candidate location that corresponds to the largest S1 intensity, the shortest pre-ejection period (Q-S1 interval), the highest occurrence rate of split S1 pattern, or the lowest occurrence rate of the paradoxical split S2 pattern, among all the candidate locations tested. In yet another example, the optimal or desired pacing location (P*) can be determined based on a difference between a paced heart sound metric and the reference heart sound metric. A candidate can be determined as the optimal or desired pacing location (P*) if the difference between the corresponding paced heart sound metric and the reference heart sound metric satisfies a specific condition, such as the S1 intensity difference, the Q-S1 interval difference, the split S1 occurrence rate difference, or the paradoxical split S2 occurrence rate difference, exceeds respective threshold amounts.

In some examples, the optimal or desired pacing location (P*) can be determined using a combination of two or more different heart sound metrics. For example, a preliminary optimal or desired pacing location or a selected subset of the potential optimal or desired pacing locations can be determined using a first heart sound metric. A different second heart sound metric can then be used to confirm the preliminary optimal or desired pacing location, or to determine the optimal or desired pacing location (P*) from the subset of the potential optimal or desired pacing locations determined based on the first heart sound metric. Using two or more heart sound metrics as such can improve the accuracy of optimal pacing site identification.

In some examples, an optimal or desired value of a pacing dosage or timing parameter can be determined using the heart sound metrics generated at 530. The optimal or desired pacing dosage and timing parameter can be determined for pacing at the optimal or desired pacing location P*. For example, changes of the paced heart sound metric at the optimal or desired pacing location P* can be determined and tracked when electrostimulation is delivered at P* with varying pacing dosage or timing parameter values. An optimal or desired pacing parameter value can be determined when the heart sound metric satisfies a specific condition, such as exceeding a threshold or falling within a predetermine value range.

At 550, the optimal or desired pacing location P* can be provided to a user (e.g., a healthcare provider), or a process such as an instance of a computer program executable in a microprocessor. The optimal or desired pacing location P*, optionally along with the corresponding heart sound metric at the location P*, may be displayed on a display screen of the user interface 330. The information may be presented in a table, a chart, a diagram, or any other types of textual, tabular, or graphical presentation formats. In some examples, the heart sound metrics (such as the S1 intensity, a cardiac timing interval such as the Q-S1 interval, the split S1 pattern, or the paradoxical split S2 pattern as described above) can be displayed in real time on a user interface while the electrostimulation is being delivered respectively to the two or more candidate locations. The real-time display of hearts sound metrics during electrostimulation, such as that shown in FIG. 4, provides a visual feedback to the user on how the different candidate locations respond to electrostimulation. In some examples, a therapy may be initiated or adjusted based on the detection of the physiological event or condition and/or the classification of the breathing pattern. Examples of the therapy may include electrostimulation therapy delivered to the heart, a nerve tissue, other target tissue, a cardioversion therapy, a defibrillation therapy, or drug therapy. For example, electrostimulation may be delivered to the optimal or desired pacing location P* in accordance with the optimal or desired value of a pacing dosage or timing parameter.

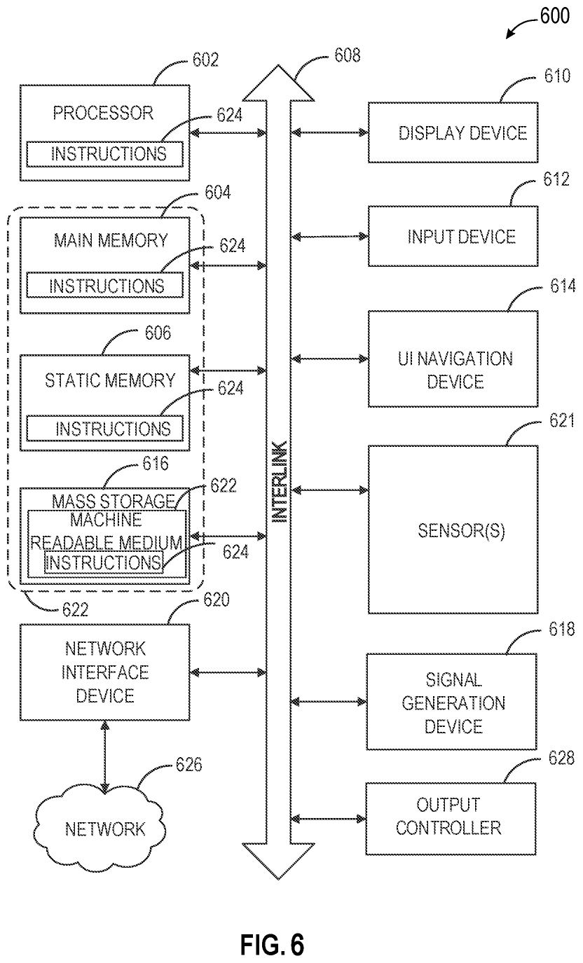

FIG. 6 illustrates generally a block diagram of an example machine 600 upon which any one or more of the techniques (e.g., methodologies) discussed herein may perform. Portions of this description may apply to the computing framework of various portions of the IMD 102, the WMD 103, the external system 105, or the heart sound-based cardiac pacing system 300.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine-readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 624 may further be transmitted or received over a communication network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communication network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should therefore be determined with references to the appended claims, along with the full scope of equivalents to which such claims are entitled.

What is claimed is:

1. A medical-device system, comprising:
a data receiver circuit configured to receive heart sound information sensed from a patient;
an electrostimulator configured to deliver electrostimulation to a location at or near a cardiac conduction system of the patient; and
a controller circuit configured to:
generate, from the heart sound information sensed from the patient in response to the electrostimulation being delivered respectively to two or more candidate locations at or near the cardiac conduction system, heart sound metrics respectively for the two or more candidate locations; and
select at least one of the candidate locations as a pacing location for subsequent electrostimulation based at least in part on the generated heart sound metrics.

2. The medical-device system of claim 1, wherein the controller circuit is configured to select the pacing location for subsequent electrostimulation from the two or more candidate locations based on a comparison of the generated heart sound metrics for the two or more candidate locations.

3. The medical-device system of claim 1, wherein the controller circuit is configured to:
generate a reference heart sound metric using heart sound information sensed from the patient without the electrostimulation being delivered to any of the two or more candidate locations; and
select the pacing location for subsequent electrostimulation that corresponds to a difference between the generated heart sound metric in response to the electrostimulation and the reference heart sound metric exceeding a threshold value.

4. The medical-device system of claim 1, wherein the electrostimulator is electrically coupled to one or more electrodes positionable at two or more candidate locations in a His-bundle or septal region of the heart to deliver the electrostimulation therein, wherein the selected pacing location for subsequent electrostimulation includes a selected His-bundle or septal site.

5. The medical-device system of claim 4, wherein the two or more candidate locations in the His-bundle or septal region include two or more candidate locations at or near one or more of a left bundle branch or a right bundle branch of the heart.

6. The medical-device system of claim 4, wherein the one or more electrodes are associated with a lead having a proximal end coupled to the electrostimulator and a distal tip configured to be inserted into the His-bundle or septal region, wherein the selected pacing location for subsequent electrostimulation includes a selected depth of insertion of the distal tip of the lead.

7. The medical-device system of claim 1, wherein the heart sound metric includes an S1 heart sound intensity,
wherein the controller circuit is configured to select the pacing location for subsequent electrostimulation with a corresponding S1 heart sound intensity exceeding a threshold.

8. The medical-device system of claim 1, wherein the data receiver circuit is configured to receive cardiac electrical activity information sensed from the patient,
wherein the controller circuit is configured to:
generate, for each of the two or more candidate locations, the heart sound metric including a pre-ejection period further using the received cardiac electrical activity information; and
select the pacing location for subsequent electrostimulation with a corresponding pre-ejection period falling below a threshold.

9. The medical-device system of claim 1, wherein the heart sound metric includes a split S1 pattern,
wherein the controller circuit is configured to select the pacing location for subsequent electrostimulation that corresponds to a detection of the split S1 pattern or an occurrence rate of the split S1 pattern exceeding a threshold.

10. The medical-device system of claim 1, wherein the data receiver circuit is configured to receive respiration information sensed from the patient, the respiration information including an inspiration phase and an expiration phase in a respiration cycle,
wherein the controller circuit is configured to:
generate, for each of the two or more candidate locations, the heart sound metric including a paradoxical split S2 pattern characterized by a presence of split S2 during the expiration phase and an absence of split S2 during the inspiration phase in the respiration cycle; and
select the pacing location for subsequent electrostimulation that corresponds to a non-detection of the paradoxical split S2 pattern or an occurrence rate of the paradoxical split S2 pattern falling below a threshold.

11. The medical-device system of claim 1, wherein the controller circuit is further configured to:
generate heart sound metrics using the heart sound information sensed from the patient in response to the electrostimulation being delivered at the selected pacing location in accordance with varying pacing dosage or timing parameter values; and
determine a pacing dosage or timing parameter value based at least in part on the generated heart sound metrics corresponding to the varying pacing dosage or timing parameter values.

12. The medical-device system of claim 1, wherein the selected pacing location is a first selected pacing location,
wherein the data receiver circuit is configured to receive cardiac electrical activity information,
wherein the controller circuit is configured to select a second pacing location from the two or more candidate locations using the received cardiac electrical activity information, and to confirm the first selected pacing location using the second selected pacing location.

13. The medical-device system of claim 1, wherein the controller circuit is configured to generate a control signal to the electrostimulator to initiate or adjust electrostimulation being delivered to the selected pacing location to restore cardiac synchrony.

14. A method of pacing a heart to restore cardiac synchrony using a medical-device system, the method comprising:
delivering electrostimulation respectively to two or more candidate locations at or near a cardiac conduction system of a patient using an electrostimulator;
receiving respective heart sound information sensed from a patient in response to the delivery of the electrostimulation at or near the two or more candidate locations;
generating heart sound metrics for the two or more candidate locations using the respective heart sound information;
selecting at least one of the candidate locations as a pacing location for subsequent electrostimulation based at least in part on the generated heart sound metrics; and
providing the selected pacing location to a user or to a process executable by the medical-device system.

15. The method of claim 14, wherein selecting the pacing location for subsequent electrostimulation is based on a comparison of the generated heart sound metrics for the two or more candidate locations.

16. The method of claim 14, comprising generating a reference heart sound metric using heart sound information sensed from the patient without the electrostimulation being delivered to any of the two or more candidate locations,
wherein that the selected pacing location corresponds to a difference between the generated heart sound metric in response to the electrostimulation and the reference heart sound metric exceeding a threshold value.

17. The method of claim 14, wherein delivering the electrostimulation respectively to the two or more candidate locations includes positioning a lead with associated one or more electrodes at or near two or more candidate locations in a His-bundle or septal region of the heart, and inserting a distal tip of the lead into the His-bundle or septal region,
wherein the selected pacing location for subsequent electrostimulation includes one or more of a selected His-bundle or septal site, or a selected depth of insertion of the distal tip of the lead.

18. The method of claim 14, wherein the heart sound metric includes at least one of:
an S1 heart sound intensity;
a pre-ejection period;
a split S1 pattern; or
a paradoxical split S2 pattern.

19. The method of claim 14, further comprising:
delivering electrostimulation at the selected pacing location in accordance with varying pacing dosage or timing parameter values;
receiving respective secondary heart sound information sensed from a patient in response to the delivery of the electrostimulation in accordance with the varying pacing dosage or timing parameter values at the selected pacing location;
generating secondary heart sound metrics using the respective secondary heart sound information; and
determining a pacing dosage or timing parameter value based at least in part on the generated secondary heart sound metrics corresponding to the varying pacing dosage or timing parameter values.

20. The method of claim 14, further comprising displaying in real time the generated heart sound metrics on a user interface while the electrostimulation is being delivered respectively to the one or more candidate location.
